SEQUENCE LISTING (8 sequences)

SEQ ID NO: 1 - DNA, 1812 bp, Homo sapiens
gaagagggcagagccgtgcatggggctgctccccaggacctgagcaggaacctggagttttcagagctgcctgatcattgctacagaatgaactctagcccagctgggaccccaagtccacagccctccagggccaatgggaacatcaacctggggccttcagccaacccaaatgcccagcccacggacttcgacttcctcaaagtcatcggcaaagggaactacgggaaggtcctactggccaagcgcaagtctgatggggcgttctatgcagtgaaggtactacagaaaaagtccatcttaaagaagaaagagcagagccacatcatggcagagcgcagtgtgcttctgaagaacgtgcggcaccccttcctcgtgggcctgcgctactccttccagacacctgagaagctctacttcgtgctcgactatgtcaacgggggagagctcttcttccacctgcagcgggagcgccggttcctggagccccgggccaggttctacgctgctgaggtggccagcgccattggctacctgcactccctcaacatcatttacagggatctgaaaccagagaacattctcttggactgccagggacacgtggtgctgacggattttggcctctgcaaggaaggtgtagagcctgaagacaccacatccacattctgtggtacccctgagtacttggcacctgaagtgcttcggaaagagccttatgatcgagcagtggactggtggtgcttgggggcagtcctctacgagatgctccatggcctgccgcccttctacagccaagatgtatcccagatgtatgagaacattctgcaccagccgctacagatccccggaggccggacagtggccgcctgtgacctcctgcaaagccttctccacaaggaccagaggcagcggctgggctccaaagcagactttcttgagattaagaaccatgtattcttcagccccataaactgggatgacctgtaccacaagaggctaactccacccttcaacccaaatgtgacaggacctgctgacttgaagcattttgacccagagttcacccaggaagctgtgtccaagtccattggctgtacccctgacactgtggccagcagctctggggcctcaagtgcattcctgggattttcttatgcgccagaggatgatgacatcttggattgctagaagagaaggacctgtgaaactactgaggccagctggtattagtaaggaattaccttcagctgctaggaagagcgactcaaactaacaatggcttcaacgagaagcaggtttattttttccagcacataaaagaaaaataatgtttcggagtccaggactggcaggacaggtcatcagatactcagaggctgtatctctgccctgccaaccttgacaaatggcttccaatgttaggtttgctacaagatggttactggagctctagctgcctattttgtgtttagggaagggaaaatggaggaaaggggagaagagcaaagggcgcttttaaagagctttcccaaaagctccccccaatgacttttgcttccatctcactaaccacccacccctacctggaatggaggctgggaaatgtggcttatttgctgggtacgtgactatccctaataacaaaggggttttgaccctaagacattaggggagaatgttgggtaggcagccagccctcttttaccatagggcctcctggtgtttggattttgatctcaatgtgtaaaatgacagagatgtaacaagctcatagggtatcaatatctcttattgttctatgttgaaaaa

SEQ ID NO: 2 - protein, 367 aa, Homp sapiens
MNSSPAGTPSPQPSRANGNINLGPSANPNAQPTDFDFLKVIGKGNYGKVLLAKRKSDGAFYAVKVLQKKSILKKKEQSHIMAERSVLLKNVRHPFLVGLRYSFQTPEKLYFVLDYVNGGELFFHLQRERRFLEPRARFYAAEVASAIGYLHSLNIIYRDLKPENILLDCQGHVVLTDFGLCKEGVEPEDTTSTFCGTPEYLAPEVLRKEPYDRAVDWWCLGAVLYEMLHGLPPFYSQDVSQMYENILHQPLQIPGGRTVAACDLLQSLLHKDQRQRLGSKADFLEIKNHVFFSPINWDDLYHKRLTPPFNPNVTGPADLKHFDPEFTQEAVSKSIGCTPDTVASSSGASSAFLGFSYAPEDDDILDC

SEQ ID NO: 3 - DNA, 28 bp, Homo sapiens
gaattccatgtcagccgaggtgcggctg

SEQ ID NO: 4 - DNA, 28 bp, Homo sapiens
gcggccgctcagggagcgcgggcggctc

SEQ ID NO: 5 - DNA, 25 bp, Homo sapiens
gtggagggcgaggaaactggggaag

SEQ ID NO: 6 - DNA, 31 bp, Homo sapiens
ctcgagtcacataatgagacagactccagtc

SEQ ID NO: 7 - protein, 13 aa, Homo sapiens
KRREILSRRPSYR

SEQ ID NO: 8 - protein, 15 aa, Homo sapiens
PLARTLSVAGLPGKK

(19) United States
(12) Patent Application Publication
(10) Pub. No.: US 2002/0123056 A1
(43) Pub. Date: Sep. 5, 2002

(54) SGK2 and its uses

(76) Inventors: Allen Delaney, Vancouver (CA); Thillainathan Yoganathan, Richmond (CA)

Correspondence Address: PAMELA J. SHERWOOD, Bozicevic, Field and Francis LLP, Suite 200, 200 Middlefield Road, Menlo Park, CA, 94025, US

(21) Appl. No.: 09/971,118

(22) Filed: Oct. 3, 2001

Related U.S. Application Data
(63) Continuation-in-part of application No. PCT/IB01/02237, filed on Sep. 20, 2001.
(60) Provisional application No. 60/237,419, filed on Oct. 2, 2000.

(51) Int. Cl. C12Q 1/68; G01N 33/574; C07H 21/04

(52) U.S. Cl. 435/6; 435/7.23; 800/3; 536/23.2

(57) ABSTRACT

Detection of SGK2 expression in cancers is useful as a diagnostic, for determining the effectiveness of drugs, and determining patient prognosis. SGK2 further provides a target for screening pharmaceutical agents effective in inhibiting the growth or metastasis of tumor cells.

### BACKGROUND

An accumulation of genetic changes underlies the development and progression of cancer, resulting in cells that differ from normal cells in their behavior, biochemistry, genetics, and microscopic appearance. Mutations in DNA that cause changes in the expression level of key proteins, or in the biological activity of proteins, are thought to be at the heart of cancer. For example, cancer can be triggered in part when genes that play a critical role in the regulation of cell division undergo mutations that lead to their over-expression.

Oncogenes are involved in the dysregulation of growth that occurs in cancers. An example of oncogene activity involves protein kinases, enzymes that help regulate many cellular activities, particularly signaling from the cell membrane to the nucleus, thus initiating the cell's entrance into the cell cycle and controlling several other functions.

Oncogenes may be tumor susceptibility genes, which are typically up-regulated in tumor cells, or may be tumor suppressor genes, which are down-regulated or absent in tumor cells. Malignancies can arise when a tumor suppressor is lost and/or an oncogene is inappropriately activated. When such mutations occur in somatic cells, they result in the growth of sporadic tumors.

Hundreds of genes have been implicated in cancer, but in most cases relationships between these genes and their effects are poorly understood. Using massively parallel gene expression analysis, scientists can now begin to connect these genes into related pathways.

Phosphorylation is important in signal transduction, a process mediated by receptors for extracellular biological signals such as growth factors or hormones. Uncontrolled signaling has been implicated in a variety of disease conditions including, inflammation, cancer, arteriosclerosis, and psoriasis. For example, many cancer causing genes (oncogenes) are protein kinases, enzymes that catalyze protein phosphorylation reactions, or are specifically regulated by phosphorylation. In addition, one kinase can have its activity regulated by one or more distinct protein kinases, resulting in specific signaling cascades.

The regulation of cell signaling events in the nucleus, for the coordinate control of target genes, allows cells to respond to external stimuli in a physiologically appropriate manner. Signal transduction pathways are crucial to the determination of the functional connections between transcriptional events regulated by nuclear steroid receptors and signaling cascades mediated by cell-surface growth factor receptors (Buse et al. (1999) J. Biol. Chem. 274:7253-63). The nuclear import of protein kinases provides one mechanism for modulating cellular signal transduction pathways.

Serum and glucocorticoid-inducible kinases (SGK) are a family of proteins in the serine/threonine protein kinase family. SGKs actively shuttle between the nucleus and the cytoplasm in synchrony with the cell cycle. SGK was originally identified as a glucocorticoid and osmotic stress-responsive gene. Two ESTs were subsequently identified with sequence similarity to the original gene. These isoforms were termed SGK2 and SGK3. In addition, there are two splice variants of SGK2; specifically, SGK2α and SGK2β. SGK2α (or β) encodes a protein of 367 residues with a calculated molecular mass of 41.1 kDa. SGK2α (or β) encodes a protein of 427 amino acids with a calculated molecular mass of 47.6 kDa. Although SGK 1, 2, and 3 share a high degree of sequence similarity, the mechanisms that regulate the level and activity of SGK2 and SGK3 differ significantly from those that regulate SGK1.

It has been shown previously that SGK1 and protein kinase B (PKB) have similar specificities towards a panel of synthetic peptides, preferentially phosphorylating Ser and Thr residues that lie in Arg-Xaa-Arg-Xaa-Xaa-Ser/Thr motifs. The specificity requirements of SGK2 and SGK3 were also found to be similar, although SGK3 appears to tolerate the presence of a Lys instead of an Arg at position n-3 (where n is the site of phosphorylation) better than SGK1 or SGK2 (Kobayashi et al. (1999) Biochemical Journal, 344: 189-197).

Because serum and glucocorticoid-induced protein kinases exert a regulatory effect on intracellular receptors by acting as transcriptional activators of genes, they play a fundamental role in the control of homeostasis, differentiation, and development in tissues. The utilization of phosphorylation-dephosphorylation networks of serine/threonine kinases by extracellular regulators, such as growth and differentiation factors, suggests that SGK2α protein kinase could play a role in the development and progression of diseases. Accordingly, there is a need in the art for improved methods for detecting and modulating the activity of SGK2α and related enzymes or kinases in vivo, and for treating diseases associated with SGKα signal transduction pathways. The present invention fulfills these needs and further provides other related advantages.

Relevant Literature

The use of genomic sequence in data mining for signaling proteins is discussed in Schultz et al. (2000) Nature Genetics 25:201. Serine/threonine protein kinases have been reviewed, for example, by Cross et al. (2000) Exp. Cell Res. 256(1):34-41. SGK2α has been described by Kobayashi et al. (1999) Biochemical Journal, 344:189-197, and by Webster et al. Molecular and Cellular Biology, (1993), 13:4, 2031-2040. Activation of SGK has been described by Kobayashi et al (1999) Biochemical Journal, 339:319-328 and in patent number WO 00/35946.

### SUMMARY OF THE INVENTION

SGK2 is shown to up-regulated in carcinoma cells. Methods are provided for the detection of SGK2 as diagnostic markers of cancer, for determining the effectiveness of drugs, and determining patient prognosis. SGK2 and its encoded protein also find utility in screening pharmaceutical agents that may act against carcinomas, in determining targets in signaling pathways involved in transformation, and as a target for therapeutic intervention in cancer.

### BRIEF DESCRIPTION OF THE DRAWINGS

FIG. 1 is a graph depicting activity of transcription factors in the presence of SGK2. AP1 and NF-κB activity was measured in HEK293 cells and in HEK293 cells stably transfected with SGK2.

FIG. 2 is a graph depicting the activation of SGK2 (K 25 plasmid) by PDK1.

### DESCRIPTION OF THE SPECIFIC EMBODIMENTS

Methods are provided for determining whether cells in a sample are cancerous. The SGK2 kinase is shown to be over-expressed in cancer cells. Detection of SGK2 over-expression in cancers provides a useful diagnostic for predicting patient prognosis and probability of drug effectiveness. Generally the amount of SGK2 detected will be compared to negative control samples from normal tissue or from known tumor cells. The presence of increased levels of SGK2 specific binding is indicative of a SGK2 associated tumor, usually at least about a 2 fold increase will be taken as a positive reaction.

SGK2 provides a target for drug screening or altering expression levels, and for determining other molecular targets involved in the kinase signal transduction pathways involved in transformation and growth of tumor cells.

The data provided herein demonstrate that SGK2α is activated by protein dependent kinase 1. CDK1 is a catalytic subunit of a protein kinase complex, called the M-phase promoting factor, that induces entry into mitosis and is universal among eukaryotes. Lee et al. (1988) Nature 333: 676-679 describe the regulated expression and phosphorylation of CDK1 in human and murine in vitro systems. Serum stimulation of human and mouse fibroblasts results in a marked increase in CDK1 transcription. Both the yeast and mammalian systems are regulated by phosphorylation of the gene product. In HeLa cells, CDK1 is the most abundant phosphotyrosine-containing protein and its phosphotyrosine content is subject to cell-cycle regulation (Draetta et al. (1988) Nature 336: 738-744). One site of CDK1 tyrosine phosphorylation in vivo is selectively phosphorylated in vitro by a product of the SRC gene. Taxol activates CDK1 kinase in MDA-MB-435 breast cancer cells, leading to cell cycle arrest at the G2/M phase and, subsequently, apoptosis. Chemical inhibitors of CDK1 block taxol-induced apoptosis in these cells (Yu et al (1998) Molec. Cell 2:581-591). Interference in this pathway is of interest in the development of therapeutic agents that affect cell cycle arrest and apoptosis.

### DIAGNOSTIC METHODS

Determination of the upregulation of SGK2 is used in the diagnosis, typing and staging of tumors. Detection of the presence of SGK2 is performed by the use of a specific binding pair member to quantitate the specific protein, DNA or RNA present in a patient sample. Generally the sample will be a biopsy or other cell sample from the tumor. Where the tumor has metastasized, blood samples may be analyzed.

### SPECIFIC BINDING MEMBERS

In a typical assay, a tissue sample, e.g. biopsy, blood sample, etc. is assayed for the presence of SGK2 specific sequences by combining the sample with a SGK2 specific binding member, and detecting directly or indirectly the presence of the complex formed between the two members. The term “specific binding member” as used herein refers to a member of a specific binding pair, i.e. two molecules where one of the molecules through chemical or physical means specifically binds to the other molecule. In this particular case one of the molecules is SGK2, where the term SGK2 is intended to include any protein substantially similar to the amino acid sequence provided in SEQ ID NO:2, or a fragment thereof; or any nucleic acid substantially similar to the nucleotide sequence provided in SEQ ID NO:1, or a fragment thereof. The complementary members of a specific binding pair are sometimes referred to as a ligand and receptor.

Binding pairs of interest include antigen and antibody specific binding pairs, peptide-MHC antigen and T-cell receptor pairs; complementary nucleotide sequences (including nucleic acid sequences used as probes and capture agents in DNA hybridization assays); kinase protein and substrate pairs; autologous monoclonal antibodies, and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Nucleic acid sequences. In another embodiment of the invention, nucleic acids are used as a specific binding member. Sequences for detection are complementary to a SGK2 sequence. The nucleic acids of the invention include nucleic acids having a high degree of sequence similarity or sequence identity to SEQ ID NO:1. Sequence identity can be determined by hybridization under stringent conditions, for example, at 50° C. or higher and 0.1XSSC (9 mM saline/0.9 mM sodium citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, etc., bind to SEQ ID NO:1 under stringent hybridization conditions.

The nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 3′ or 5′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression in tumor cells.

Probes specific to the nucleic acid of the invention can be generated using the nucleic acid sequence disclosed in SEQ ID NO:1. The probes are preferably at least about 18 nt, 25 nt, 50 nt or more of the corresponding contiguous sequence of SEQ ID NO:1, and are usually less than about 2, 1, or 0.5 kb in length. Preferably, probes are designed based on a contiguous sequence that remains unmasked following application of a masking program for masking low complexity, e.g. BLASTX. Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

For hybridization probes, it may be desirable to use nucleic acid analogs, in order to improve the stability and binding affinity. The term “nucleic acid” shall be understood to encompass such analogs. A number of modifications have been described in the art that alter the chemistry of the phosphodiester backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Antibodies. The SGK2 polypeptides of the invention may be used for the production of antibodies, where short fragments provide for antibodies specific for the particular polypeptide, and larger fragments or the entire protein allow for the production of antibodies over the surface of the polypeptide. As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions, is a term well understood in the art, and indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a SGK2 polypeptide, particularly a human SGK2 polypeptide.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

### METHODS FOR QUANTITATION OF NUCLEIC ACIDS

Nucleic acid reagents derived from the sequence of SGK2 are used to screen patient samples, e.g. biopsy-derived tumors, inflammatory samples such as arthritic synovium, etc., for amplified SGK2 DNA, or increased expression of SGK2 mRNA or protein. DNA-based reagents are also designed for evaluation of chromosomal loci implicated in certain diseases e.g. for use in loss-of-heterozygosity (LOH) studies, or design of primers based on SGK2 coding sequence.

The polynucleotides of the invention can be used to detect differences in expression levels between two cells, e.g., as a method to identify abnormal or diseased tissue in a human. The tissue suspected of being abnormal or diseased can be derived from a different tissue type of the human, but preferably it is derived from the same tissue type; for example, an intestinal polyp or other abnormal growth should be compared with normal intestinal tissue. The normal tissue can be the same tissue as that of the test sample, or any normal tissue of the patient, especially those that express the polynucleotide-related gene of interest (e.g., brain, thymus, testis, heart, prostate, placenta, spleen, small intestine, skeletal muscle, pancreas, and the mucosal lining of the colon, etc.). A difference between the polynucleotide-related gene, mRNA, or protein in the two tissues compared, for example, a difference in molecular weight, amino acid or nucleotide sequence, or relative abundance, indicates a change in the gene, or a gene which regulates it, in the tissue of the human that was suspected of being diseased.

The subject nucleic acid and/or polypeptide compositions may be used to analyze a patient sample for the presence of polymorphisms associated with a disease state. Biochemical studies may be performed to determine whether a sequence polymorphism in a SGK2 coding region or control regions is associated with disease, particularly cancers and other growth abnormalities. Diseases of interest may also include other hyperproliferative disorders. Disease associated polymorphisms may include deletion or truncation of the gene, mutations that alter expression level, that affect the binding activity of the protein, the kinase activity domain, etc.

Changes in the promoter or enhancer sequence that may affect expression levels of SGK2 can be compared to expression levels of the normal allele by various methods known in the art. Methods for determining promoter or enhancer strength include quantitation of the expressed natural protein; insertion of the variant control element into a vector with a reporter gene such as beta-galactosidase, luciferase, chloramphenicol acetyltransferase, etc. that provides for convenient quantitation; and the like.

A number of methods are available for analyzing nucleic acids for the presence of a specific sequence, e.g. upregulated expression. Cells that express SGK2 may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp.14.2-14.33.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2,7-dimethoxy-4,5-dichloro-6-c arboxyfluorescein (JOE), 6-carboxy-X-rhodamine (ROX), 6-carboxy-2,4,7,4,7-hexachlorofluorescein (HEX), 5-carboxyfluorescein (5-FAM) or N, N, N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. Probes may be hybridized to Northern or dot blots, or liquid hybridization reactions performed. The nucleic acid may be sequenced by dideoxy or other methods, and the sequence of bases compared to a wild-type SGK2 sequence. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

Arrays provide a high throughput technique that can assay a large number of polynucleotides in a sample. In one aspect of the invention, an array is constructed comprising SGK2, which array may further comprise other sequences known to be up-or down-regulated in tumor cells. This technology can be used as a tool to test for differential expression.

A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Samples of nucleic acids can be detectably labeled (e.g., using radioactive or fluorescent labels) and then hybridized to the probes. Double stranded nucleic acids, comprising the labeled sample polynucleotides bound to probe nucleic acids, can be detected once the unbound portion of the sample is washed away. Alternatively, the nucleic acids of the test sample can be immobilized on the array, and the probes detectably labeled.

Techniques for constructing arrays and methods of using these arrays are described in, for example, Schena et al. (1996) Proc. Natl. Acad. Sci. U.S.A. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

Arrays can be used to, for example, examine differential expression of genes and can be used to determine gene function. For example, arrays can be used to detect differential expression of SGK2, where expression is compared between a test cell and control cell (e.g., cancer cells and normal cells). High expression of a particular message in a cancer cell, which is not observed in a corresponding normal cell, indicates a cancer specific gene product. Exemplary uses of arrays are further described in, for example, Pappalarado et al. (1998), Sem. Radiation Oncol. 8:217; and Ramsay (1998), Nature Biotechnol. 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

Polypeptide Analysis

Screening for SGK2 may be based on the functional or antigenic characteristics of the protein. Protein truncation assays are useful in detecting deletions that may affect the biological activity of the protein. Various immunoassays designed to detect polymorphisms in SGK2 proteins may be used in screening. Where many diverse genetic mutations lead to a particular disease phenotype, functional protein assays have proven to be effective screening tools. The activity of the encoded SGK2 protein in kinase assays, etc., may be determined by comparison with the wild-type protein.

A sample is taken from a patient with cancer. Samples, as used herein, include biological fluids such as blood; organ or tissue culture derived fluids; etc. Biopsy samples or other sources of carcinoma cells are of particular interest, e.g. tumor biopsy, etc. Also included in the term are derivatives and fractions of such cells and fluids. The number of cells in a sample will generally be at least about 10³, usually at least 10⁴, and may be about 10⁵ or more. The cells may be dissociated, in the case of solid tissues, or tissue sections may be analyzed. Alternatively a lysate of the cells may be prepared.

Detection may utilize staining of cells or histological sections, performed in accordance with conventional methods. The antibodies or other specific binding members of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and SGK2 in a lysate. Measuring the concentration of SGK2 binding in a sample or fraction thereof may be accomplished by a variety of specific assays. A conventional sandwich type assay may be used. For example, a sandwich assay may first attach SGK2 specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These plates are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of SGK2 is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute nonionic detergent medium at an appropriate pH, generally 7-8, is used as a wash medium. From one to six washes may be employed, with sufficient volume to thoroughly wash away non-specifically bound proteins present in the sample.

After washing, a solution containing a second antibody is applied. The antibody will bind SGK2 with sufficient specificity such that it can be distinguished from other components present. The second antibodies may be labeled to facilitate direct, or indirect quantification of binding. Examples of labels that permit direct measurement of second receptor binding include radiolabels, such as ³H or ¹²⁵l, fluorescers, dyes, beads, chemilumninescers, colloidal particles, and the like. Examples of labels that permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules; generally from about 0.1 to 3 hr, and most commonly usually 1 hr.

After the second binding step, the insoluble support is again washed free of nonspecifically bound material, leaving the specific complex formed between SGK2 and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for SGK2 as desired, conveniently using a labeling method as described for the sandwich assay.

In some cases, a competitive assay will be used. In addition to the patient sample, a competitor to SGK2 is added to the reaction mix. The competitor and the SGK2 compete for binding to the specific binding partner. Usually, the competitor molecule will be labeled and detected as previously described, where the amount of competitor binding will be proportional to the amount of SGK2 present. The concentration of competitor molecule will be from about 10 times the maximum anticipated SGK2 concentration to about equal concentration in order to make the most sensitive and linear range of detection.

In some embodiments, the methods are adapted for use in vivo, e.g., to locate or identify sites where cancer cells are present. In these embodiments, a detectably-labeled moiety, e.g., an antibody, which is specific for SGK2 is administered to an individual (e.g., by injection), and labeled cells are located using standard imaging techniques, including, but not limited to, magnetic resonance imaging, computed tomography scanning, and the like. In this manner, cancer cells are differentially labeled.

The detection methods can be provided as part of a kit. Thus, the invention further provides kits for detecting the presence of an mRNA encoding SGK2, and/or a polypeptide encoded thereby, in a biological sample. Procedures using these kits can be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

Samples for Analysis

Samples of interest include tumor tissue, e.g., excisions, biopsies, blood samples where the tumor is metastatic, etc. Of particular interest are solid tumors, e.g. carcinomas, and include, without limitation, tumors of the liver and colon. Liver cancers of interest include hepatocellular carcinoma (primary liver cancer). Also called hepatoma, this is the most common form of primary liver cancer. Other liver cancers of interest for analysis by the subject methods include hepatocellular adenoma, which are benign tumors occuring most often in women of childbearing age; hemangioma, which are a type of benign tumor comprising a mass of abnormal blood vessels, cholangiocarcinoma, which originates in the lining of the bile channels in the liver or in the bile ducts; hepatoblastoma, which is common in infants and children; angiosarcoma, which is a rare cancer that originates in the blood vessels of the liver; bile duct carcinoma and liver cysts. Cancers originating in the lung, breast, colon, pancreas and stomach and blood cells commonly are found in the liver after they become metastatic.

Also of interest are colon cancers. Types of cancer of the colon and rectum include polyps, which are any mass of tissue that arises from the bowel wall and protrudes into the lumen. Polyps may be sessile or pedunculated and vary considerably in size. Such lesions are classified histologically as tubular adenomas, tubulovillous adenomas (villoglandular polyps), villous (papillary) adenomas (with or without adenocarcinoma), hyperplastic polyps, hamartomas, juvenile polyps, polypoid carcinomas, pseudopolyps, lipomas, leiomyomas, or other rare tumors.

### SCREENING METHODS

Target Screening

The availability of a number of components in signaling pathways allows in vitro reconstruction of the pathway, and/or assessment of kinase action on targets. Two or more of the components may be combined in vitro, and the behavior assessed in terms of activation of transcription of specific target sequences; modification of protein components, e.g. proteolytic processing, phosphorylation, methylation, etc.; ability of different protein components to bind to each other etc. The components may be modified by sequence deletion, substitution, etc. to determine the functional role of specific domains.

The SGK2 specific reagents are used to identify targets of SGK2 in cancers. For example, SGK2 may be introduced into a tumor cell using an inducible expression system. Suitable positive and negative controls are included. Transient transfection assays, e.g. using adenovirus vectors, may be performed. The cell system allows a comparison of the pattern of gene expression in transformed cells with or without SGK2 expression. Alternatively, phosphorylation patterns after induction of SGK2 are examined. Gene expression of putative target genes may be monitored by Northern blot or by probing microarrays of candidate genes with the test sample and a negative control where SGK2 is not induced. Patterns of phosphorylation may be monitored by incubation of the cells or lysate with labeled phosphate, followed by 1 or 2 dimensional protein gel analysis, and identification of the targets by MALDI, micro-sequencing, Western blot analysis, etc., as known in the art.

Some of the potential target genes of SGK2 identified by this method will be secondary or tertiary in a complex cascade of gene expression or signaling induced by SGK2. To identify primary targets of SGK2 activation, expression or phosphorylation will be examined early after SGK2 induction (within 1-2 hours) or after blocking later steps in the cascade with cycloheximide.

Target genes or proteins identified by this method may be analyzed for expression in primary patient samples as well. The data for SGK2 and target gene expression may be analyzed using statistical analysis to establish a correlation between SGK2 and target gene expression.

### COMPOUND SCREENING

Compound screening may be performed using an in vitro model, a genetically altered cell or animal, or purified SGK2 protein. One can identify ligands or substrates that bind to, modulate or mimic the action of SGK2. Areas of investigation include the development of treatments for hyper-proliferative disorders, e.g. cancer, restenosis, osteoarthritis, metastasis, etc.

The polypeptides include those encoded by SEQ ID NO:1, as well as nucleic acids that, by virtue of the degeneracy of the genetic code, are not identical in sequence to the disclosed nucleic acids, and variants thereof. Variant polypeptides can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by SEQ ID NO:1, or a homolog thereof.

Transgenic animals or cells derived therefrom are also used in compound screening. Transgenic animals may be made through homologous recombination, where the normal SGK2 locus is altered. Alternatively, a nucleic acid construct is randomly integrated into the genome. Vectors for stable integration include plasmids, retroviruses and other animal viruses, YACs, and the like. A series of small deletions and/or substitutions may be made in the SGK2 gene to determine the role of different exons in kinase activity, oncogenesis, signal transduction, etc. Of interest is the use of SGK2 to construct transgenic animal models for cancer, where expression of SGK2 is specifically reduced or absent or present in multiple copies and is more than normally expressed. Specific constructs of interest include antisense SGK2, which will block SGK2 expression and expression of dominant negative SGK2 mutations. A detectable marker, such as lac Z may be introduced into the SGK2 locus, where up-regulation of SGK2 expression will result in an easily detected change in phenotype. One may also provide for expression of the SGK2 gene or variants thereof in cells or tissues where it is not normally expressed or at abnormal times of development. By providing expression of SGK2 protein in cells in which it is not normally produced, one can induce changes in cell behavior, e.g. in the control of cell growth and tumorigenesis.

Compound screening identifies agents that modulate SGK2 function. Agents that mimic its function are predicted to activate the process of cell division and growth. Conversely, agents that inhibit SGK2 function may inhibit transformation. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of SGK2, derived from crystallization of purified recombinant SGK2 protein, could lead to the rational design of small drugs that specifically inhibit SGK2 activity. These drugs may be directed at specific domains of SGK2, e.g. the kinase catalytic domain, the regulatory domain, the auto-inhibitory domain, etc.

The term “agent” as used herein describes any molecule, e.g. protein or pharmaceutical, with the capability of altering or mimicking the physiological function of SGK2. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. A mixture of such components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Other assays of interest detect agents that mimic SGK2 function. For example, an expression construct comprising a SGK2 gene may be introduced into a cell line under conditions that allow expression. The level of SGK2 activity is determined by a functional assay, for example detection of protein phosphorylation. Alternatively, candidate agents are added to a cell that lacks functional SGK2, and screened for the ability to reproduce SGK2 in a functional assay.

The compounds having the desired pharmacological activity may be administered in a physiologically acceptable carrier to a host for treatment of cancer, etc. The compounds may also be used to enhance SGK2 function in wound healing, cell growth, etc. The inhibitory agents may be administered in a variety of ways, orally, topically, parenterally e.g. subcutaneously, intraperitoneally, by viral infection, intravascularly, etc. Depending upon the manner of introduction, the compounds may be formulated in a variety of ways. The concentration of therapeutically active compound in the formulation may vary from about 0.1-10 wt %.

### FORMULATIONS

The compounds of this invention can be incorporated into a variety of formulations for therapeutic administration. Particularly, agents that modulate SGK2 activity, or SGK2α polypeptides and analogs thereof are formulated for administration to patients for the treatment of cells where the SGK2 activity is undesirably high or low, e.g. to reduce the level of SGK2 in cancer cells. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intra-tracheal, etc., administration. The SGK2 may be systemic after administration or may be localized by the use of an implant that acts to retain the active dose at the site of implantation.

In pharmaceutical dosage forms, the compounds may be administered in the form of their pharmaceutically acceptable salts, or they may also be used alone or in appropriate association, as well as in combination with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well known in the art. Implants are formulated as microspheres, slabs, etc. with biodegradable or non-biodegradable polymers. For example, polymers of lactic acid and/or glycolic acid form an erodible polymer that is well tolerated by the host. The implant is placed in proximity to the site of disease, so that the local concentration of active agent is increased relative to the rest of the body.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic lipids, such as phosphatidylcholine. The remaining lipid will normally be neutral lipids, such as cholesterol, phosphatidyl serine, phosphatidyl glycerol, and the like. Polyethylene glycol is often used to change the pharmacokinetics of injectable liposomal formulations.

For preparing the liposomes, the procedure described by Kato et al. (1991) J. Biol. Chem. 266:3361 may be used. Briefly, the lipids and lumen composition containing the nucleic acids are combined in an appropriate aqueous medium, conveniently a saline medium where the total solids will be in the range of about 1-10 weight percent. After intense agitation for short periods of time, from about 5-60 sec., the tube is placed in a warm water bath, from about 25-40° C. and this cycle repeated from about 5-10 times. The composition is then sonicated for a convenient period of time, generally from about 1-10 sec. and may be further agitated by vortexing. The volume is then expanded by adding aqueous medium, generally increasing the volume by about from 1-2 fold, followed by shaking and cooling. This method allows for the incorporation into the lumen of high molecular weight molecules.

### MODULATION OF SGK2 ACTIVITY

Agents that block SGK2 activity provide a point of intervention in an important signaling pathway. Numerous agents are useful in reducing SGK2 activity, including agents that directly modulate SGK2 expression as described above, e.g. expression vectors, antisense specific for SGK2; and agents that act on the SGK2 protein, e.g. SGK2 specific antibodies and analogs thereof, small organic molecules that block SGK2 catalytic activity, etc.

The SGK2 gene, gene fragments, or the encoded protein or protein fragments are useful in therapy to treat disorders associated with SGK2 defects. From a therapeutic point of view, inhibiting SGK2 activity has a therapeutic effect on a number of proliferative disorders, including inflammation, restenosis, and cancer. Inhibition is achieved in a number of ways. Antisense SGK2 sequences may be administered to inhibit expression. Pseudosubstrate inhibitors, for example, a peptide that mimics a substrate for SGK2 may be used to inhibit activity. Other inhibitors are identified by screening for biological activity in an SGK2 based functional assay, e.g. in vitro or in vivo SGK2 kinase activity.

Expression vectors may be used to introduce the SGK2 gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or SGK2 protein may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold micro projectiles are coated with the SGK2α or DNA, then bombarded into skin cells.

Antisense molecules can be used to down-regulate expression of SGK2 in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to antisense inhibitors, catalytic nucleic acid compounds, e.g. ribozymes, antisense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 95/23225, and Beigelman et al. (1995) Nucl. Acids Res. 23:4434-42). Examples of oligonucleotides with catalytic activity are described in WO 95/06764. Conjugates of antisense ODN with a metal complex, e.g. terpyridyl Cu(II), capable of mediating mRNA hydrolysis are described in Bashkin et al. (1995) Appl. Biochem Biotechnol. 54:43-56.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLE 1

### SGK2

The Genbank database was searched for ESTs showing similarity to known kinase domain-related proteins using the “basic local alignment search tool” program, TBLASTN, with default settings. Human ESTs identified as having similarity to these known kinase domain (defined as p<0.0001) were used in a BLASTN and BLASTX screen of the GenBank non-redundant (NR) database.

ESTs that had top human hits with >95% identity over 100 amino acids were discarded. This was based upon the inventors' experience that these sequences were usually identical to the starting probe sequences, with the differences due to sequence error. The remaining BLASTN and BLASTX outputs for each EST were examined manually, i.e., ESTs were removed from the analysis if the inventors determined that the variation from the known kinase domain-related probe sequence was a result of poor database sequence. Poor database sequence was usually identified as a number of ‘N’ nucleotides in the database sequence for a BLASTN search and as a base deletion or insertion in the database sequence, resulting in a peptide frameshift, for a BLASTX output. ESTs for which the highest scoring match was to non-kinase domain-related sequences were also discarded at this stage.

Using widely known algorithms, e.g. “Smith/Waterman”, “Fasta”, “FastP”, “Needleman/Wunsch”, “Blast”, “PSIBlast,” homology of the subject nucleic acid to other known nucleic acids was determined. A “Local FastP Search” algorithm was performed in order to determine the homology of the subject nucleic acid invention to known sequences. Then, a ktup value, typically ranging from 1 to 3 and a segment length value, typically ranging from 20 to 200, were selected as parameters. Next, an array of position for the probe sequence was constructed in which the cells of the array contain a list of positions of that substring of length ktup. For each subsequence in the position array, the target sequence was matched and augmented the score array cell corresponding to the diagonal defined by the target position and the probe subsequence position. A list was then generated and sorted by score and report. The criterion for perfect matches and for mismatches was based on the statistics properties of that algorithm and that database, typically the values were: 98% or more match over 200 nucleotides would constitute a match; and any mismatch in 20 nucleotides would constitute a mismatch. Analysis of the BLASTN and BLASTX outputs identified a EST sequence from IMAGE clone AF169034 that had potential for being associated with a sequence encoding a kinase domain-related protein, e.g., the sequence had homology, but not identity, to known kinase domain-related proteins.

The AF169034 IMAGE clone was sequenced using standard ABI dye-primer and dye-terminator chemistry on a 377 automatic DNA sequencer. Sequencing revealed that the sequence corresponds to SEQ ID NO:1, SGK2α. The expression of SGK2α was determined by dot blot analysis, and the protein was found to be upregulated in several tumor samples. SEQ ID NO:3 and 4 were used in amplification.

Dot blot preparation. Total RNA was purified from clinical cancer and control samples taken from the same patient. Samples were used from both liver and colon cancer samples. Using reverse transcriptase, cDNAs were synthesized from these RNAs. Radiolabeled cDNA was synthesized using Strip-EZ™ kit (Ambion, Austin, Tex.) according to the manufacturer's instructions. These labeled, amplified cDNAs were then used as a probe, to hybridize to human protein kinase arrays comprising human SGK2. The amount of radiolabeled probe hybridized to each arrayed EST clone was detected using phosphorimaging.

The expression of SGK2 was substantially upregulated in the tumor tissues that were tested. The data is shown in Table 1, expressed at the fold increase over the control non-tumor sample.

TABLE 1

| | | | | colon | colon | colon | colon | colon | colon | colon |
|---|---|---|---|---|---|---|---|---|---|---|
| | liver 1 | liver 2 | liver 3 | 1 | 4 | 5 | 7 | 8 | 9 | 10 |
| SGK2 | 3.6 | 2.4 | 1.1 | 1.1 | 1.0 | 3.9 | 1.8 | 1.4 | 0.7 | 2.55 |
| beta-actin | 2.05 | 1.07 | 1.57 | 0.42 | 1.28 | 2.19 | 1.20 | 4.60 | 0.60 | 0.49 |
| GAPDH | 1.30 | 0.33 | 1.25 | 0.76 | Not | Not | Not | Not | Not | Not |
| | | | | | done | done | done | done | done | done |
| K413 (ribosomal | Not | Not | Not | Not | 1.72 | 2.36 | 2.10 | 1.00 | 1.00 | 1.68 |
| protein) | done | done | done | done | | | | | | |

The data displayed in Table 2 provides a brief summary of the pathology report of the patient samples.

TABLE 2

| | | | Precursor | Site of | | Vascular | Lymphatic | |
|---|---|---|---|---|---|---|---|---|
| Patient | Age | Gender | Adenoma | Involvement | Differentiation | Invasion | Involvement | Metastasis |
| Liver 1 | 49 | Female | N/a | Liver | Moderately | No | Yes | No |
| | | | | | Differentiated | | | |
| Liver 2 | 53 | Male | N/a | Liver | Moderately | Yes | No | No |
| | | | | | Differentiated | | | |
| Liver 3 | 75 | Female | Yes | Right Colon | Moderately | No | No | No |
| | | | | | Differentiated | | | |
| Colon | 55 | Female | No | Rectum | Moderately | N/A | Yes | No |
| 1 | | | | | Differentiated | | | |
| Colon | 91 | Female | Yes | Cecum | Moderately | No | Yes | No |
| 4 | | | | | Differentiated | | | |
| Colon | 79 | Male | No | Ileum and | Moderately | No | No | No |
| 5 | | | | Colon | Differentiated | | | |
| Colon | 93 | Male | No | Rectosigmoid | Moderately | No | No | No |
| 7 | | | | | Differentiated | | | |
| Colon | 61 | Male | Yes | Yes | Moderately | No | Yes | Yes, Liver |
| 8 | | | | | Differentiated | | | |
| Colon | 60 | Male | No | Recto- | Moderately | Yes | No | Yes, Liver |
| 9 | | | | Sigmoid | Differentiated | | | |
| Colon | 60 | Male | No | Sigmoid | Moderately | Yes | Yes | No |
| 10 | | | | Colon | Differentiated | | | |

Creation of stable cell lines over expressing SGK2 in HEK293 cells. We constructed a mammalian expression vector encoding N-terminal X-press tagged forms of the 45 kDa SGK2 kinase. The ORF of SGK2 was placed in frame with N-terminal Xpress and a Histidine tag in pcDNA 3 mammalian expression vector using standard PCR-based cloning techniques. To characterize SGK2 at the protein level, HEK293 cells were transfected and a stable cell line selected with pcDNA 3 His-X-press-SGK2 plasmid in the presence of G418. HEK293 cells were stably transfected with mammalian vector incorporating SGK2 to produce clones over expressing wild type SGK2.

Briefly, cells were grown in d-MEM containing 5% FCS, 2 mm L-glutamine, glucose (3.6 mg/ml) and G418 (40 μg/ml) was added to transfected cells to maintain selection pressure. The cell lysates were prepared from stable cell lines and subjected to Western blotting with anti-Xpress mAb and anti-His-antibody. A protein with a 45 kDa molecular mass was identified in lysates of HEK293 cells stably expressing SGK2. A similar protein could not be detected in the control HEK293 cells. This analysis suggests that HEK293 cells are overexpressing SGK2 as a fusion protein. To determine whether these cells express higher levels of SGK2 mRNA, we isolated mRNA from stable cell lines as well as control HEK293 cells. Equal amounts of mRNA were immobilized on a nylon membrane and subjected to hybridization with a specific SGK2 probe. Stable cell lines expressed a significantly higher concentration of SGK2 mRNA as compared to control HEK293 cells. These results indicate that stable cell lines are over expressing SGK2 mRNA as well as SGK2 protein. These stable cell lines were used in the subsequent experiments.

Overexpressed SGK2 can phosphorylate GSK3 in vivo. We explored the identification of the downstream effectors of SGK2 by using SGK2 ovexpressing cells. SGKs have 54% nucleotide sequence homology to PKB and it has previously been shown that PKB could phosphorylate GSK3 in vivo and in vitro. In view of this, we wanted to determine whether SGK2 could regulate the activity of GSK3, a kinase that is normally phosphorylates beta catenin. GSK3 phosphorylates beta catenin and targets it for destruction via a ubiquitinproteasome pathway. To determine whether SGK2 could phosphorylate GSK3, initially, we carried out transient transfection assays in human embryonal kidney epithelial cells (HEK293). Transfection of SGK2 resulted in increased phosphorylation of GSK3. This was monitored by specific anti-GSK3 phospho Ser9 antibody. These results suggest that SGK2 effects the phosphorylation of GSK3 in vivo.

As a control, we measured the concentration of GSK3 protein in the assay. The concentration of GSK3 is not affected by SGK2 but the phosphorylation status of GSK3 is affected by the expression of SGK2. This is particularly significant at the lower concentration of serum (0.5%) and 0.1-0.2 μg concentration of SGK2 plasmid. Because GSK3 activity can be inhibited by phosphorylation, it is possible that inhibition of GSK3 by SGK2 could lead to other downstream effects. To further evaluate the link between SGK2 and GSK3 we measured the phosphorylation status of GSK3 in HEK293 cells and in HEK293 cells stably transfected with SGK2 (named SGK-37A). SGK-37A cells overexpressing SGK2 had significantly higher phospho GSK3 than normal HEK293 cells.

This data demonstrates that SGK2 can modulate the phosphorylation status of GSK3 in stably transfected HEK293 cells. It has been shown that GSK3 phosphorylation leads to GSK3 inactivation (Cross et al. (1995) Nature 378:785-789). SGK2 may directly phosphorylate GSK3 and inactivate it, thereby abolishing phosphorylation of the cytoplasmic signaling molecule β-catenin and causing its stabilization and nuclear translocation. In the nucleus, β-catenin associates with TCF4 to induce the transcription of several genes including cyclin D1.

SGK2 enhances cell proliferation. Since we have shown that overexpression of SGK2 stimulates GSK3 phosphorylation, it was investigated whether this could lead to cell proliferation. To study the effects of SGK2 on cell proliferation, we used several cells types. These cells were transiently transfected with SGK2 or control DNA plasmids. The DNA synthesis rate was determined by measuring [³H] thymidine incorporation. When HEK293 and 3T3 cells were transfected with SGK2, they exhibited greater amounts of DNA synthesis than the control vector. The rate of proliferation was dependent on the concentration of transfected SGK2 plasmid. This data indicates that SGK2 stimulates cell proliferation in these cell types. Co-expression of PDK1 with SGK further enhanced the rate of proliferation.

These data reveal that SGK2 promotes proliferation in a variety of cells, and suggest that SGK2 promotes cell proliferation and support tumor progression in these types of cells.

SGK overexpression stimulates AP1 transactivation. It has previously been shown that GSK3 phosphorylates c-Jun at C-terminal sites, resulting in inhibition of DNA binding (Nikolakaki et al. (1993) Oncogene 8:833-840) This can lead to the inhibition of AP1 activity in intact cells. It is believed that this keeps the cell's homeostasis in control. Since we have shown that SGK2 phosphorylates GSK3, we wanted to evaluate whether this could modulate the AP1 transactivation in cells overexpressing SGK2.

AP1 activity was measured in HEK293 cells and in HEK293 cells stably transfected with SGK2. SGK-37A clones have been shown to overexpress SGK2. AP1 activity was several fold higher in SGK-37A than in control HEK293 cells (FIG. 3). This data suggests that SGK2 can upregulate AP1 promoter activity in HEK293 cells. In the nucleus, AP1 transactivation induces the transcription of several genes involved in proliferation and several MMP genes. Our data suggests that SGK2 can induce an invasive phenotype via AP1 dependent upregulation of MMP gene expression.

SGK2 stimulates the translocation of beta catenin into the nucleus. SGK2 stabilizes beta catenin in HEK293 cells. To determine whether overexpression of SGK2 in HEK293 cells would induce beta catenin stability, we employed immunocytochemistry analysis. Monoclonal antibody for beta catenin was used in the analysis. In vivo expression of beta catenin was measured by standard protocols. The results indicate that SGK2 expressing cells have a higher concentration of beta catenin than parental cells. β catenin is localized entirely in the nucleus of SGK2 overexpressing cells, suggesting that SGK2 regulates the translocation of beta catenin into the nucleus.

Taken together, these results indicate that SGK2 is an important intracellular regulator of signaling via components of the Wnt/wingless pathway, specifically through modulation of GSK3β activity. Beta catenin has a consensus sequence phosphorylation site for GSK3β, and GSK3β acts to cause the degradation of beta catenin. Several studies have shown that GSK3β phosphorylates β catenin and that the phosphorylation of β catenin is essential for its degradation. If β catenin is not phosphorylated, the stability of β catenin increases in the cytoplasm and subsequently increases the translocation of beta catenin to the nucleus. In the nucleus, beta catenin associates with TCF4 to induce the transcription of several genes including cyclin D1.

SGK stimulates TCF4 transcriptional activity. The nuclear translocation of beta catenin is associated with a complex formation between β catenin and members of the high mobility group transcription factors, LEF1/TCF, which then activate transcription of target genes. LEF1 is a transcription factor that is by itself unable to stimulate transcription from multimerized sites, although in association with β catenin LEF1/TCF proteins can augment promoter activity from multimerized binding sites.

We examined the transcriptional activation of a synthetic TCF4/β catenin responsive promoter construct containing TCF4 binding sites in HEK293 cells overexpressing SGK2 and in control HEK293 cells. Higher promoter activity was observed only in SGK2 overexpressing cells. Transient transfection of increasing concentrations of TCF4 reporter gene produced concentration dependent TCF4 transactivation in SGK2 over expressing cells, whereas transient transfection of TCF4 reporter gene into HEK293 cells did not produce significant transactivation. This result indicates that SGK2 selectively targets GSK3β. Regulated β catenin subsequently increased the TCF4 transactivation in HEK293 cells. These data indicates that SGK2 overexpression overcomes the regulation of TCF4 expression by adhesion/deadhesion, and that it maintains constitutively high levels of TCF4 transactivation. TCF4/β catenin has been shown to induce transcription of genes encoding homeobox proteins that regulate mesenchymal genes,and this pathway is likely to mediate the epithelial to mesenchymal transformation. Constitutive activation of TCF/β catenin is oncogenic in human colon carcinomas. The data presented here show that SGK2 can modulate β catenin signaling and transactivate TCF4 reporter genes.

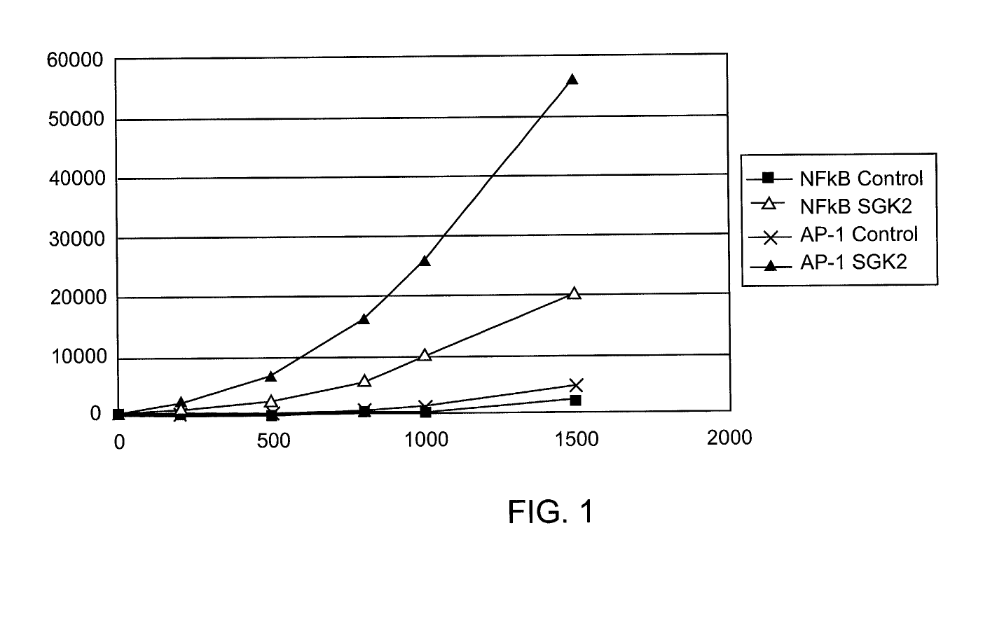

SGK2 stimulate NF—kB transcription. It has previously been shown that PKB/AKT regulate NF—κB mediated transactivation. In view of this, we next asked whether SGK2 could activate the NF—κB reporter assay in vivo. To evaluate NF—κB transactivation, the NF—κB promoter containing luciferase plasmid was transiently transfected into HEK293 cells overexpressing SGK2 and control HEK293 cells. As shown in FIG. 1, the activity of the NF—κB reporter was several fold higher in SGK2 overexpressing cells than in control HEK293 cells. Increasing concentration of NF—κB reporter plasmid in the SGK2 overexpressing cells increased luciferase activity, whereas NF—κB mediated transactivation had no significant effect on the control HEK293 cell. This data demonstrates that SGK2 can regulate NF—κB transactivation.

NF—kB transactivation occurs in response to the major proapoptotic signals, including TNF-α, anticancer drugs, and ionizing radiation. Several reports have indicated that in some cancer cell types, NF—κB is an important factor for cell survival. Hence, SGK2 may promote cell survival in certain cell types and participate in tumor promotion.

NF—κB DNA binding activity coincides with degradation of IκB alpha. To examine the status of IκB alpha in the SGK2 overexpressing cells, we performed the following experiment. Cellular extracts were made from HEK293 cells overexpressing SGK2 and control HEK293 cells. These cell extracts were analyzed against a specific anti-phospho IκB alpha antibody. Increasing concentrations of cell extract produced increasing IκB alpha phospho signal, whereas the same protein concentration of control HEK293 cell extracts did not produce IκB alpha phospho signals. These results suggest that NF—κB activation by SGK2 is mediated by IκB alpha phosphorylation.

SGK2 phosphorylation of BAD. SGK2 phosphorylates some of the proteins phosphorylated by PKB. It has previously been shown that PKB can phosphorylate BAD. It was tested whether SGK2 phosphorylates BAD. Protein was isolated from HEK293 cells overexpressing SGK2 and control HEK293 cells; and the phosphorylation status of BAD was measured. The cells were lysed and the expression of BAD phosphorylation was determined by anti-BAD phospho antibody. SGK2 overexpressing cells contain higher levels of phospho Bad protein than normal cells, although expression levels of BAD protein were unaffected by SGK2. These finding show that SGK2 increases BAD phosphorylation in HEK293 cells.

Phosphorylation of BAD may lead to the prevention of cell death via a mechanism that involves the selective association of phosphorylated forms of BAD with 14-3-3 protein isoforms. The identification of BAD as a SGK2 substrate expands the list of in vivo SGK2 targets. Recent studies have revealed that BAD represents a point of convergence of several different signal transduction pathways that are activated by survival factors that inhibit apoptosis in mammalian cells. These data suggest that SGK2 inhibits apoptosis in mammalian cells through phosphorylation of BAD.

Phosphorylation of FKHR in HEK293 cells. The forkhead family of transcription factors is involved in tumorigenesis in rhabodomyosarcoma and acute leukemias. FKHR, FKHRL1, and AFX mediate signaling via a pathway involving IGFR1, PI3K and PKB/AKT. Phosphorylation of FKHR family members by PKB/AKT promotes cell survival and regulates FKHR nuclear translocation and target gene transcription. Insulin stimulation specifically promotes phosphorylation of this threonine site and causes FKHR cytoplasmic retention by 14-3-3 protein binding on the phosphorylated sequence.

To investigate whether FKHR could be phosphorylated by SGK2 in a cellular context, we created HEK293 cells stably expressing SGK2 and then examined FKHR phosphorylation with phospho specific antibodies. These experiments demonstrated that FKHR, Thr24 or Ser 256 were phosphorylated at low levels in normal HEK293 cells whereas HEK293 stable cells had higher levels of FKHR phosphorylation. This data shows that FKHR exhibits higher phosphorylation status in SGK2 overexpressing cells.

It has previously been shown that FKHR phosphorylation leads to FKHR's interaction with 14-3-3 proteins and sequestration in the cytoplasm, away from its transcriptional targets. The unphosphorylated FKHR accumulates in the nucleus where it activates death genes, including Fas ligand gene, and thereby participates in the process of apoptosis. Upon phosphorylation, FKHR interacts with 14-3-3 and is retained in the cytoplasm thereby inhibiting its ability to activate transcription. Therefore, phosphorylation of FKHR by SGK2 can promote cell survival.

CREB phosphorylation is regulated by SGK2. To determine whether CREB is a regulatory target for SGK2, we performed the following experiments. Equal amounts of protein were isolated from SGK2 overexpressing cells as well as control HEK293 cells and subjected to phospho CREB analysis. The cells were lysed and the amount of CREB phosphorylation was determined by CREB phospho (Ser133) antibody. SGK2 overexpressing cells contain higher levels of phospho CREB protein than normal cells, showing that SGK2 increases CREB phosphorylation

Studies by have indicated that CREB function is important in promoting cell survival. Cyclin D1 expression is regulated by CREB. The majority of breast cancer cell lines and mammary tumors overexpress cyclin D1, suggesting that induction of cyclin D1 may play an important role in mammary tumorigenesis. These studies further clarify the mechanism by which SGK2 could promote cell survival. CREB function is important in promoting cell survival by responding to growth factor stimulation. These data imply that SGK2 modulates the phosphorylation status of CREB in vivo, and therefore is involved in cell survival through the CREB pathway.

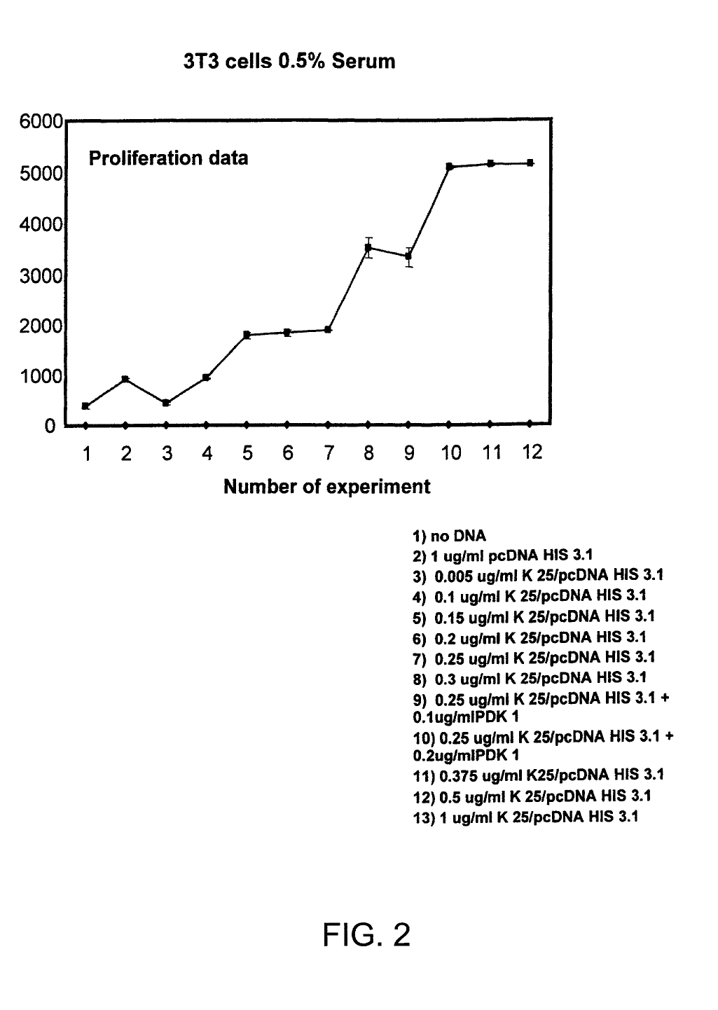

SGK2 is activated by PDK1 and the activation leads to increased kinase activity. To determine whether cloned and purified SGK2 can phosphorylate specific peptides directly, SGK2 was purified from insect cells. Activation was performed in vitro by mixing SGK2 and PDK1. After the activation, the PDK1 was removed from the mixture and purified SGK2 was used for the analysis. The cell extracts were purified by GST affinity column chromatography and the purity was analyzed by SDS-PAGE. Both non-activated and PDK1-activated SGK2 produced similar amounts of protein. SGK2 activated by PKD1 was significantly phosphorylated, while non-activated SGK2 was not. The data is shown in FIG. 2.

The kinase activity of SGK2 was evaluated using specific peptides. SGK2 was incubated with two different peptide substrates ((SEQ ID NO: 32) PKB-sub: CKRPRAASFAE; and (SEQ ID NO:33) PDK1: KTFCGTPEYLAPEV RREPRILS EEEQEMFRDFDYI (UBI Catalogue #12401), and in vitro kinase assays carried out. Equivalent concentration of purified SGK2 were incubated using a Beckman Biomek 2000 robotic system. Each well containing 25μl reaction mixture composed of 10μl SGK2, 5μl of assay dilution buffer, 5 μl of peptide substrate and 5 μl of γ ³²P—ATP. The kinase reaction was carried out for 15 minutes at room temperature (22° C.). At the end of the reaction period, 10 μl of the reaction mixture was spotted onto 96-well p81 phosphocellulose multiscreen plates from Millipore, washed and the ³²P incorporation was counted in a Wallac Microbeta scintillation counter.

Peptides incubated with purified SGK 2 gave significant incorporation of ³²P, whereas in the absence of peptides no significant incorporation was seen. When comparing the peptides, PKB-sub had significant incorporation of ³²P whereas addition of same amount of control peptide (PDK1 peptide) had no significant incorporation. This data demonstrates that purified SGK2 possesses a kinase activity in vitro. Moreover, the PDK1 activated SGK2 had significantly higher kinase activity compared to non-activated SGK2. These data clearly demonstrate that activated SGK2 phosphorylates the GSK3 Ser9 (GSK3β consensus) sequence, supporting the previous observation that SGK2 overexpressing cells exhibit higher level of GSK3 Ser9 phosphorylation than control cells.

SGK2 kinase activity is stimulated by Calyculin A and Okadaic acid. Hi5 insect cells expressing GST-SGK2 were treated with 100 nM microcysteine, 99.8 nM okadaic acid and 49.8 nM calyculin A for four hours at 27° C. The GST-SGK2 fusion protein was purified by GST-agarose affinity column and eluted with 20 mM Glutathione/50 mM Tris-HCl/50 mM NaCl, pH 7.5. Substrates were PKB sub and CapK sub at 1 mg/ml, for 15 minutes at room temperature. The results were as follows:

| | No-Substrate | | |
|---|---|---|---|
| | (CCPM1) | PKB sub (CCPM1) | CapK sub(CCPM1) |
| Untreated | 349 | 979 | 1081 |
| Microcysteine | 305 | 217 | 330 |
| Calyculin A | 0 | 92540 | 59335 |
| Okadaic Acid | 2078 | 132171 | 161553 |

These data indicate that okadaic acid and Calyculin A stimulated SGK2 kinase activity, suggesting that okadaic and Calyculin A can stimulate SGK2 activity. It has previously been shown that protein phosphatase inhibitors such as okadaic acid and Calyculin A modulate phosphorylation of several nuclear proteins.

These findings demonstrate SGK2 could promote cell survival and cell growth. Overexpression of SGK2 in HEK293 cells increased GSK3 phosphorylation thereby inhibiting the activity of GSK3, and subsequently leading to AP1 transactivation. GSK3 is involved in regulation of several intracellular signaling pathways, of which the Wnt pathway is of particular interest. In mammals, Wnt signaling increases the stability of beta catenin resulting in transcriptional activation of LEF-1/TCF. In SGK2 overexpressing cells we have shown increased LEF-1/TCF transactivation through increasing the stability of the beta catenin pool in the cells, suggesting that SGK2 activates the Wnt signaling pathway, which can lead to nuclear localization of beta catenin and increased transactivation of LEF-1/TCF.

At least 6 SGK2 substrates have been identified in mammalian cells, and they fall into two main classes: regulators of apoptosis and regulators of cell growth, including protein synthesis and glycogen metabolis.. The SGK2 substrates involved in cell/death regulation include Forkhead transcription factors (FKHR), the pro-apoptotic Bcl-2 family member BAD, and the cyclic AMP response element binding protein (CREB).

We have also demonstrated that SGK2 could regulate signaling pathways that lead to induction of the NF—κB family of transcription factors in HEK293 cells. This induction occurs at the level of degradation of the NF—κB inhibitor IκB and is specific for NF—κB. These data suggest that SGK2 appears to be a point of convergence for several different signaling pathways. Taken together, our results suggest that the over expression of SGK2 may therefore play a central role in tumor cell progression.

Materials and Methods.

Dulbecco's Modified Eagle Medium (DMEM), RPMI Medium 1640, L-glutamine, phosphate buffered solution (PBS), fetal bovine serum (FBS), and restriction enzymes were from GibcoBRL. TOPO cloning kit (including PCR materials and pCR 2.1-Topo vector) were from Invitrogen. Phospho-CREB (Ser133) polygonal rabbit antibody was from Cell Signaling Technology. 96-and 6-well delta surface plates were from NUNCLON. QlAprep Spin Miniprep Kit was from Qiagen. Wizard Plus Minipreps DNA Purification System (for gel extractions) (Promega). FuGENE 6 Transfection Reagent was from Boehringer Mannheim. pcDNA3.1 mammalian expression vector (Invitrogen). Western Blotting Luminol Reagent was from Santa Cruz Biotechnology. 2° goat-anti-rabbit IgG (H+L) HRP conjugated antibody was from Bio-Rad Laboratories.

Cloning of full length SGK2. To generate the full length cDNA of SGK2, a pair of primers were designed and used in a PCR reaction. The amplification product was cloned through restriction sites, EcoR I and Xho I, into bacteria expression vector pGEX-4T-3 and mammalian expression vector pcDNA3.1/His B. All construct were verified by restriction enzyme digestion and DNA sequencing.

Cell Culture. Cells were incubated at 37° C. in 5% CO₂ (standard conditions). All cells, unless mentioned below, were cultured in DMEM with FBS; the specific amount of FBS varies and is stated in the report for each result. Jurkat cells were cultured in RPMI Medium 1640 with added glucose, L-glutamine, and 10% FBS.

Cell Transfection. Cells were seeded to a density of 2×10⁵ in 6 well plates (in appropriate media for the particular cell line) and incubated for 24 hours under standard conditions. 3 ml of FuGENE 6 transfection reagent was diluted in 97 ml of serum-free media (appropriate for the cell line being transfected) and left for 5 minutes at room temperature; that was then added drop-wise to the desired amount of plasmid DNA (in pcDNA3.1) and left for 10 minutes at room temperature. The finished transfection solution was then added dropwise to the cells, which were then incubated for 24 hours under standard conditions.

Proliferation Assay. The media from 6 well plates was removed and trypsin was added to digest the extracellular matrix holding the cells to the plate; media (appropriate to the cell type) was then added to deactivate the trypsin. The cells and media were transferred into Falcon tubes, centrifuged, and the supernatant was discarded. The cells were resuspended in appropriate media. 3000 cells were seeded in each well of a 96 well plate and appropriate media was added up to 90 ml. Ten μl of 0.1 Ci/L ³H-thymidine was added to each well. The plates were then incubated for 24 hours under standard conditions. Twentyfive μl of cold trichloroacetic acid was added to each well and the plates were kept at 4° C. for 2 hours. The plates were then washed in cold running water and allowed to dry. Proliferation was determined by incorporation of thymidine as measured via scintillation counting.

Cell lysis. Lysis buffer was 50 mM Hepes (pH 7.5), 150 mM NaCl, 1% NP-40, 2 mM NaF, 1 mM Na₃VO₄, 1 mM PMSF, 1 mg/ml pepstatin, 1 mg/ml leupeptin, 1 mg/ml aprotinin, and 20 mM β-glycerophosphate. For adherent cells, the media was removed from the 6 well plate, the wells were washed with PBS which was then removed, the plates were put on ice and 40 ml of lysis buffer was then added to each well. Crude lysates were collected with a cell scraper and placed in an Eppendorf tube. For non-adherent cells, the media and cells were transferred from a 6-well plate to tubes, centrifuged and the supernatant removed; 40 ml of lysis buffer was then added. All crude lysates were then vortexed and left on ice for 10 minutes. The crude lysates were centrifuged at 14,000 RPM for 10 minutes at 4° C. and the supernatant, the final lysate, was transferred to new tubes.

Western Blotting. Equal weights of cell lysate proteins were mixed with 4X loading buffer, boiled for five minutes and were then briefly centrifuged. The samples were run on a 10% SDS-PAGE and were then transferred to PVDF membranes which were washed with TTBS and blocked with 2% BSA. They were blotted with primary antibody for 16 hours at 4° C. The membranes were washed with TTBS, blotted with secondary antibody for 1 hour and washed with TTBS. The luminol reagent was added, the blot was placed on film and the autoradiograph developed.

Expression and Purification of SGK2 Protein. The human SGK2 gene was subcloned into baculovirus transfer vector pAcG2T (BD PharMingen) under the control of the strong AcNPV (Autograpga californica Nuclear Polyhedrosis Virus) polyhedrin promoter. This was co-transfected with linear BaculoGoldTM DNA in Spodoptera frugiperda Sf9 cells following the manufacturer's procedure (BD PharMingen). The high titer of GST-SGK2 recombinant baculovirus was amplified in Sf9 cells in TNM-FH medium (JHR Biosciences) with 10% fetal bovine serum. The GST-SGK2 protein was expressed in about 5×10⁸ Hi5 cells (Invitrogen) in 500 ml of Excell-400 medium (JHR Biosciences) with about 5 MOl for a period of 72 h in a spinner flask. The cells were harvested at 800Xg for 5 min at 4° C. The pellet was lysed in 40 ml of Lysis Buffer by sonication and centrifuged at 10,000Xg at 4° C. for 15 min. The supernatant was loaded on the column contained 2.5 ml of glutathione-agarose (Sigma). The column was washed with Wash Buffer A until OD280 returned to baseline, then Wash Buffer B. The GST-SGK2 protein was eluted in Elution Buffer. The fraction was aliquoted and stored at −70° C.

Assay of SGK2. SGK2 was assayed at room temperature for 15 min with 25 μl of reaction mixture containing 5 mM MOPS, PH7.2, 5 mM MgCl₂, 5 mM β-glycerophosphate, 50 μM dithiothreitol, 1 μM β-methyl aspartic acid, 1 mM EGTA, 0.5 mM EDTA, 0.5 μM PKI, 50 μM [γ-³²P]-ATP and 0.2 μg/ul PKB-sub peptide (UBI) or PDKtide peptide (UBI) as substrates. GSK3 consensus peptide (SEQ ID NO:5, PKB-sub: CKRPRAASFAE), PDK1 sub-SEQ ID NO:6, KTFCGTPEYLAPEVRREPRILSEEEQEMFRDFDYI. Reactions were initiated by addition of [γ-³²P]-ATP and terminated by spotting 10 μl of aliquots onto cellulose phosphate paper in 96-well filtration plate (Millipore), followed by washing in 1% phosphoric acid. The dried plate was added 25 μl scintillant (Amersham) and counted.

SGK2 Phosphorylation by PDK1. SGK2 was incubated with active His-tag PDK1 in the presence of Mg²⁺/ATP. His-tag PDK1 was expressed in insect cells and purified on Talon affinity column. SGK2 phosphorylation assay was performed at room temperature for 20 min in 25 μl of reaction solution consisting of 10 mM MOPS, PH 7.2, 15 mM MgCl₂, 5 mM β-glycerophosphate, 1 mM EGTA, 0.2 mM sodium orthovanadate, 0.2 mM dithiothreitol, 0.5 μM PKI, 0.2 μM Microcystin-LR, 75 ng/μl Ptdlns (3,4,5) P3 (PIP3), 156 ng/μl dioleoyl phosphatidylcholine (DOPC), 156 ng/μl dioleoyl phosphatidylserine (DOPS), 50 μM [γ-³²P]-ATP, ˜20 ng His-PDK1 and ˜5 μg GST-SGK2. The reaction were incubated and terminated by addition of 25 μl 2X loading buffer. No PDK1 was added to negative control reaction. 25 μl of above loading samples were run on 9% SDS-PAGE. The dried Coomassia blue-stained gel was imaged in GS-525 Molecular Imagera System (BIO-RAD).

SGK2 Activation by PDK1. About 2.5 mg of GST-SGK2 and 1 μg of His-PDK1 were incubated at 4° C. for 2 hours in 20 ml of activation solution containing 10 mM MOPS, PH 7.2, 15 mM MgCl₂, 5 mM β-glycerophosphate, 1 mM EGTA, 0.2 mM sodium orthovanadate, 0.2 mM dithiothreitol, 0.5 μM PKI, 0.2 μM Microcystin-LR, 75 ng/μl Ptdlns (3,4,5) P3 (PIP3), 156 ng/μl dioleoyl phosphatidylcholine (DOPC), 156 ng/μl dioleoyl phosphatidylserine (DOPS), and 10 mM ATP. The glutathione was removed from the activation solution on Q-sepharose column. The activated GST-SGK2 were re-purified from glutathione-agarose column.

Cell and cell culture. 293 cells were stably transfected with a mammalian vector incorporating SGK2 to produce overexpressing wild type SGK2. Cells were grown in MEM containing 10% FCS, 2 mm L-glutamine, glucose (3.6 mg/ml), insulin (10 μg/ml), and G418 (40 μg/ul) were added to transfected cells to maintain selection pressure.

Transient transfection: HEK293 cells were seeded at 1.5×10⁵ cells/well plate and grown for 24 hr before transfection. Various concentration of plasmid DNA were transfected using Fugene (Roche) according to the manufucture's protocol. DNA content was normalized with appropriate empty expression vectors. Cells were starved for O/N in DMEM containing 0.5% FBS.

Western blotting: Cells were lysed for 10 minutes on ice in NP-40 lysis buffer (1% NP40, 50 mM Hepes, pH 7.4, 150 mM Nacl, 2 mM EDTD, 2 mM PMSF, 1 mM Na-o-vanadate, 1 mM NaF, 10 μg/ml aprotinin, 10 μg/ml leupeptin). Extracts were centrifuged with the resulting supernatants being the cell lysate used in assays. Lysates were electrophoresed through SDS-PAGE and transferred to Immobilin-P (Millipore Bedford, MD). Antibodies used to probe Western blots were: Anti-Xpresss, Phospho-FKHR (Thr24, Caspase-9, Phospho-IkBalpha (Ser32/36), Bad, Phospho CREB, Phospho GSK3 alpha (ser-9), GSK3 monoclonal, (New England Biolab, Mississauga, ON, Canada) Bands were visualized with ECL chemiluminescent substrate (Amersham Pharmacia biotech).

Reporter assay: 293 cells were transfected in 6-well plates with Fugene (Roche Diagnostics) according to the manufacture's instructions. To analyse various reporter assay, respective reporter construct were transiently transfected with indicated amount of luciferase reporter gene construct series of LEF-1/TCF binding sites, AP1 binding sites and NF—κB binding sites. Extracts were prepared and assayed 24-48 after transfection and relative luciferase activity was determined using Promega Dual luciferase reporter assay system as described by the manufacture.

Immunocytochemistry: 293-cell lines were grown in 8 chamber slides for 2 days, washed with PBS, fixed in absolute cold methanol for 10 minutes, washed with PBS and incubated overnight at 4° C. with beta-catenin (#C19220-BD Transduction Laboratories), His —Prob (#Sc-803, Santa Cruz, USA) and anti-Xpress antibody (R910-25, Invitrogen), all diluted 1:100 in PBS with 0.1% Triton X-100, then washed with PBS. Proceed with immunostaining by using the ABC method (ABC-Elite kit, Vector). Acccording to the amount and intensity of staining, the scale was divided into 2 classes. The slides designated “+” had positive staining intensity, slides designated “−” showed no immunoreactivity. In addition to conventional light microscopic examination, in order to quantitate the amount of reactivity, specimens were also investigated by computerized image analysis using Image pro (Media Cybernetics, MD, USA).

Expression and Purification of GST-SGK2 from Hi 5 Insect cells. Human SGK2 was cloned into the Baculovirus vector pAcG2T with the multiple cloning sites in the vector.. This vector contains an N-terminal Glutathione S-transferase tag (GST-tag) which allows for affinity purification on Glutathione agarose beads. The vector was infected into Sf9 insect cells via lipid vesicles. The titer of the baculovirus particles was amplified in Sf9 insect cells. The amplified baculovirus titer was then used to infect four 250 ml volume spinner-flasks (Pyrex) containing Hi 5 cells which were at approximately 0.8×10⁶ cells/ml. The expression of the fusion protein cells were incubated at 27° C., with spinning at 80 rpm, over 3.5 days. Near the end of this expression period, each of the four 180 ml cultures of Hi 5 cells were stimulated with a 4 hour, 27° C. treatment with either 100% DMSO (negative control) or one of three different PP1 and PP2a phosphatase inhibitors: 100 nM Microcystin (Calbiochem), 55.05 nM Calyculin A (Calbiochem), and 96.9 nM Okadaic Acid (Calbiochem). Finally, the cells were collected by centrifugation in Beckman Avant-25 rotor ID 10.500 at 3000 rpm, 5 min, 4° C. After a brief 1×PBS wash, the cells were resuspended in a 50 mM Tris-HCl/1% NP-40, pH 7.5 lysis buffer supplemented with the following protease inhibitors: 100 μM Sodium Vanadate, 1 mM glycerophosphate, and 237 μl Protease Inhibitor Cocktail Set III (Calbiochem). The cells were lysed using the small probe of the sonic dismembrator: output 1:3 repititions of 8 sec on and 5 sec pause. Once the cytosolic proteins are released into the supernatant, the cellular debris is removed by centrifugation in Beckman Avanti-30: 14,000 rpm, 15 min, 4° C. The lysate supernatant is applied to Glutathione-agarose beads (SIGMA) and allowed to batch-bind, rotating end-over-end, at 4° C. for 30 mins. Non-specific proteins are washed from the beads 5 times with STEL 500 (50 mM Tris-HCl/500 mM NaCl, pH 7.5) followed by 5 times with STEL 50 (50 mM Tris-HCl/50 mM NaCl, pH 7.5). Finally, the GSTtagged fusion protein is eluted from the beads with Elution buffer (20 mM glutathione/50 mM Tris-HCl/50 mM NaCl). Purified SGK2 kinase activity is assayed against PKB peptide SEQ ID NO:7 (CKRPRAASFAE), a universal SRC kinase family substrate and CapK peptide SEQ ID NO:8 (CGRTGRRNSI).

What is claimed is:

1. A method of screening for biologically active agents that modulate a cancer associated protein kinase function, the method comprising: combining a candidate biologically active agent with any one of:
(a) a polypeptide encoded by SEQ ID NO:1;
(b) a cell comprising a nucleic acid encoding a polypeptide encoded by SEQ ID NO:1; or
(c) a non-human transgenic animal model for cancer associated kinase gene function comprising one of: (i) a knockout of a gene corresponding SEQ ID NO:1; (ii) an exogenous and stably transmitted mammalian gene sequence comprising polypeptide encoded by SEQ ID NO:1; and
determining the effect of said agent on kinase function.

2. A method for the diagnosis of cancer, the method comprising:
determining the upregulation of expression of SEQ ID NO:1 in said cancer.

3. The method of claim 2, wherein said cancer is a liver cancer.

4. The method of claim 2, wherein said cancer is a colon cancer.

5. The method of claim 2, wherein said determining comprises detecting the presence of increased amounts of mRNA in said cancer.

6. The method of claim 2, wherein said determining comprises detecting the presence of increased amounts of protein in said cancer.

7. A method for inhibiting the growth of a cancer cell, the method comprising downregulating activity of the polypeptide encoded by SEQ ID NO:1; in said cancer cell.

8. The method according to claim 7, wherein said method comprises introducing antisense sequences specific for SEQ ID NO: 1.

9. The method according to claim 7, wherein said method comprises introducing an inhibitor of kinase activity into said cancer cell.

10. The method according to claim 7, wherein said cancer cell is a liver cancer cell.

11. The method according to claim 7, wherein said cancer cell is a colon cancer cell.

12. A method of screening for targets of a cancer associated protein kinase, wherein said targets are associated with signal transduction in cancer cells, the method comprising:
comparing the pattern of gene expression in a normal cell, and in a tumor cell characterized by up-regulation of SEQ ID NO:1.

13. The method according to claim 12, wherein said comparing the pattern of gene expression comprises quantitating specific mRNAs by hybridization to an array of polynucleotide probes.

14. A method of screening for targets of a cancer associated protein kinase, wherein said targets are associated with signal transduction in cancer cells, the method comprising:
comparing the pattern of protein phosphorylation in a normal cell, and in a tumor cell characterized by up-regulation of SEQ ID NO:1.

15. The method according to claim 12, wherein said signal transduction involves activation by protein dependent kinase 1.

16. The method according to claim 14, wherein said signal transduction involves activation by protein dependent kinase 1.

17. An isolated nucleic acid comprising the sequence set forth in SEQ ID NO:1.
